(19) United States
(12) Patent Application Publication
(10) Pub. No.: US 2010/0055108 A1
(43) Pub. Date: Mar. 4, 2010

(54) DRUG FOR TREATING GASTRIC CANCER

(75) Inventors: Pierre Miossec, Bron (FR); Ling Toh, Lyon (FR)

Correspondence Address: OLIFF & BERRIDGE, PLC, P.O. BOX 320850, ALEXANDRIA, VA, 22320-4850, US

(73) Assignees: BIOMERIEUX, MARCY L'ETOILE (FR); HOSPICES CIVILS DE LYON, LYON (FR)

(21) Appl. No.: 12/312,774

(22) PCT Filed: Dec. 20, 2007
(86) PCT No.: PCT/FR2007/052575

§ 371 Date: May 27, 2009

(30) Foreign Application Priority Data
Dec. 21, 2006 (FR) 0655835

(51) Int. Cl. A61K 39/395; C07K 16/00; C07H 21/00; A61K 31/7052; A61P 35/00

(52) U.S. Cl. 424/158.1; 530/389.2; 536/24.5; 514/44 A

(57) ABSTRACT

The invention relates to the use of at least one interleukin-17 inhibitor and/or of at least one IL-17 receptor inhibitor, for the manufacture of a medicament for inhibiting, preventing or treating gastric cancer.

The present invention relates to gastric cancer, and more particularly to a new treatment for combating this cancer.

Stomach cancer is the second most common cancer throughout the world, and accounts for approximately 10% of the cases of cancer. Most of the individuals who are the most susceptible to contracting this pathological condition have a diet which is rich in starch, and low in fat, in proteins, in fruit and in fresh vegetables. Salt, alcohol and an excessive consumption of smoked foods are also implicated. An infection due to Helicobacter pylori is also often implicated.

At the beginning, stomach cancer causes only few symptoms, which are often not very characteristic, making early diagnosis even more difficult. When it exists, the most common symptom is pain located in the upper and medium part of the abdomen. Sometimes, the patient is subject to losing weight, vomiting, anemia and/or the presence of blood in the stools. The diagnosis is generally made by endoscopy or fibroscopy, which make it possible to detect small cancerous tumors at an early stage, tumors which are sometimes benign, or other lesions present in the gastric mucosa.

The treatment for curative purposes is generally a partial or total gastrectomy. In the case of early-stage cancer (“chronic atrophic gastritis”), the eradication of H. pylori is also important. There is currently no chemotherapy protocol that is the subject of a consensus. The improvement obtained with respect to survival and quality of life compared with a simple symptomatic treatment is moderate.

It is therefore important, from a clinical point of view, to propose new treatment alternatives to the clinician.

The present invention proposes to solve the drawbacks of the prior art by providing new biological tools for improving the diagnosis and the treatment of a patient for gastric cancer.

The inventors have demonstrated that interleukin-17F (IL-17F), just like interleukin-17A (IL-17A), plays a very important role in gastric cancer.

In this respect, the invention concerns the use of at least one interleukin-17 inhibitor and/or of at least one IL-17 receptor inhibitor, for the manufacture of a medicament for inhibiting, preventing or treating gastric cancer.

According to one preferred embodiment of the invention, the interleukin-17 is interleukin-17A (IL-17A) or interleukin-17F (IL-17F).

According to one preferred embodiment of the invention, said IL-17 receptor is the IL-17 receptor A or the IL 17 receptor C.

According to one preferred embodiment of the invention, the interleukin-17 inhibitor is an antibody directed against IL-17, preferably against IL-17A or F, and/or the inhibitor of said IL-17 receptor is an antibody directed against the IL-17 receptor, preferably against the IL-17 receptor A or C.

According to another preferred embodiment of the invention, the interleukin-17 inhibitor is an interfering RNA against IL-17, preferably against IL-17A or F, and/or the inhibitor of said IL-17 receptor is an interfering RNA against the IL-17 receptor, preferably against the IL-17 receptor A or C.

The invention also concerns a pharmaceutical composition comprising, as active substance, at least one interleukin-17 inhibitor and/or at least one IL-17 receptor inhibitor in combination with a pharmaceutically appropriate carrier.

According to one preferred embodiment of the invention, the interleukin-17 is interleukin-17A (IL-17A) or interleukin-17F (IL-17F).

According to one preferred embodiment of the invention, said IL-17 receptor is the IL-17 receptor A or the IL-17 receptor C.

According to one preferred embodiment of the invention, the interleukin-17 inhibitor is an antibody directed against IL-17, preferably against IL-17A or F, and/or the inhibitor of said IL-17 receptor is an antibody directed against the IL-17 receptor, preferably against the IL-17 receptor A or C.

According to one preferred embodiment of the invention, the interleukin-17 inhibitor is an interfering RNA against IL-17, preferably against IL-17A or F, and/or the inhibitor of said IL-17 receptor is an interfering RNA against the IL-17 receptor, preferably against the IL-17 receptor A or C.

The invention also concerns the use of the pharmaceutical composition as defined above, for inhibiting, preventing and/or treating gastric cancer.

The following definitions will make it possible to understand the invention more clearly.

The term “IL-17 receptor” is intended to mean a molecule of the IL-17 receptor family, said receptors being defined by their likeness to the IL-17RA receptor (Moseley T. A. et al., 2003, Cytokine Growth Factor Rev, 14(2): 155-74). Preferably, said IL-17 receptor is the IL-17 receptor A or the IL-17 receptor C.

The term “IL-17RA receptor” is intended to mean the molecule initially discovered for its involvement in the inflammatory and/or immunostimulant activity of IL-17A (Yao Z. et al., 1997, Cytokine, 9(11): 794-800).

The term “IL-17RC receptor” is intended to mean an IL-17RA receptor-like molecule (Haudenschild D. et al., 2002, J Biol Chem, 277: 4309-4316).

The term “interleukin-17F inhibitor” is intended to mean a molecule (or a collection of molecules) which block(s) the inflammatory and/or immunostimulant activity of IL-17F.

The inhibitor may in particular be an antibody directed against IL-17F, or an interfering RNA against IL-17.

The term “IL-17 receptor inhibitor” is intended to mean a molecule (or a collection of molecules) which block(s) the action of an IL-17 receptor.

The inhibitor may in particular be an antibody directed against the IL-17 receptor, preferably against the IL-17 receptor A or C. The inhibitor may also be an interfering RNA against the IL-17 receptor, preferably against the IL-17 receptor A or C.

The term “gastric cancer” is intended to mean any stomach cancer.

The term “antibody” is intended to mean both a whole antibody and an antibody fragment.

The recombinant antibodies can be obtained according to conventional methods known to those skilled in the art, using prokaryotic organisms, such as bacteria, or using eukaryotic organisms, such as yeasts, mammalian cells, plant cells, insect cells or animal cells, or by means of extracellular production systems.

The monoclonal antibodies may be prepared according to the conventional techniques known to those skilled in the art, such as the hybridoma technique, the general principle of which is summarized below.

Firstly, an animal, generally a mouse (or cells in culture in the case of in vitro immunizations) is immunized with a target antigen of interest, and the B lymphocytes of said animal are then capable of producing antibodies against said antigen. These antibody-producing lymphocytes are subsequently fused with “immortal” myeloma cells (murine in the example) so as to produce hybridomas. Using the heterogeneous cell mixture thus obtained, a selection of the cells capable of producing a particular antibody and of multiplying indefinitely is then carried out. Each hybridoma is multiplied in the form of a clone, each resulting in the production of a monoclonal antibody of which the recognition properties with respect to the antigen of interest may be tested, for example, by ELISA, by one- or two-dimensional immunoblotting, by immunofluorescence, or using a biosensor. The monoclonal antibodies thus selected are subsequently purified, in particular according to the affinity chromatography technique.

Antibody fragments can, for example, be obtained by proteolysis. Thus, they can be obtained by enzymatic digestion, resulting in fragments of Fab type (treatment with papain; Porter R R, 1959, Biochem. J., 73: 119-126) or of F(ab)′2 type (treatment with pepsin; Nisonoff A. et al., 1960, Science, 132: 1770-1771). They can also be prepared by the recombinant process (Skerra A., 1993, Curr. Opin. Immunol., 5: 256-262). Another antibody fragment which is suitable for the purposes of the invention comprises an Fv fragment, which is a dimer constituted of the noncovalent association of the variable light (VL) domain and of the variable heavy (VH) domain of the Fab fragment, and therefore the association of two polypeptide chains. In order to improve the stability of the Fv fragment due to the dissociation of the two polypeptide chains, this Fv fragment can be modified by genetic engineering, by inserting a suitable peptide linker between the VL domain and the VH domain (Huston P. et al., 1988, Proc. Natl. Acad. Sci. USA, 85: 5879-5883). This is then referred to as an scFv fragment (single chain Fragment variable) since it is made up of a single polypeptide chain. The use of a peptide linker composed preferentially of 15 to 25 amino acids makes it possible to link the C-terminal end of one domain to the N-terminal end of the other domain, thus constituting a monomeric molecule having binding properties similar to those of the antibody in its complete form. Both orientations of the VL and VH domains are suitable (VL-linker-VH and VH-linker-VL) since they have identical functional properties. Of course, any fragment known to those skilled in the art and having the immunological characteristics defined above is suitable for the purposes of the invention.

Preferably, the antibody is directed against IL-17A or F or against the IL-17 receptor, preferably against the IL-17 receptor A or C.

The term “interfering RNA” is intended to mean a ribonucleic acid which blocks the expression of a given gene (Dallas A. et al., 2006, Med Sci Monit, 12(4): RA67-74). Preferably, the iRNA interferes with IL-17, preferably IL-17A or F, or with the IL-17 receptor, preferably the IL-17 receptor A or C.

The term “medicament” or “pharmaceutical composition” is intended to mean any substance or composition presented as having curative or preventive properties with regard to human or animal diseases, and also any product that can be administered to humans or to animals with a view to establishing a medical diagnosis or to restoring, correcting or modifying their organic functions.

The term “active substance” is intended to mean a compound acknowledged as having therapeutic properties.

In the pharmaceutical compositions according to the invention, for oral, sublingual, subcutaneous, intramuscular, intravenous, topical, intratracheal, rectal or transdermal administration, the active substances may be administered in unit administration forms or as a mixture with conventional pharmaceutical carriers, which are intended for oral administration, for example in the form of a tablet, a gel capsule, an oral solution, etc, or rectal administration, in the form of a suppository, parenteral administration, in particular in the form of an injectable solution, especially intravenous, intradermal, subcutaneous, etc., administration, according to conventional protocols well known to those skilled in the art. For topical application, the active substances can be used in creams, ointments or lotions.

When a solid composition in tablet form is prepared, the active substances are mixed with a pharmaceutically acceptable excipient, also known as pharmaceutically appropriate carrier, such as gelatin, starch, lactose, magnesium stearate, talc, gum arabic, or the like. The tablets can be coated with sucrose, a cellulosic derivative, or other suitable substances. They can also be treated in such a way that they have a sustained or delayed activity and that they continuously release a predetermined amount of active substances. It is also possible to obtain a preparation of gel capsules by mixing the active substances with a diluent and by pouring the mixture into soft or hard gel capsules. It is also possible to obtain a preparation in syrup form or for administration in the form of drops, in which the active substances are present together with a sweetener, an antiseptic, for instance methylparaben and propylparaben, and also a flavor enhancer or a suitable dye. Water-dispersible powders or granules can contain the active substances as a mixture with dispersing agents or wetting agents, or suspending agents, well known to those skilled in the art. For parenteral administration, aqueous suspensions, isotonic saline solutions or injectable sterile solutions which contain dispersing agents, and pharmacologically compatible wetting agents, such as in particular propylene glycol or butylene glycol, are used.

The medicament or the pharmaceutical composition according to the invention may also comprise an activating agent which induces the effects of a medication or reinforces or supplements the effects of the principal medication, by increasing in particular the bioavailability of the principal medication.

The dosage depends on the seriousness of the condition. In the case of a pharmaceutical composition comprising an antibody, the administration may in particular be carried out once every 2 to 8 weeks, preferably with 50 to 100 mg of antibody, in combination with a pharmaceutically acceptable excipient. In the case of a pharmaceutical composition comprising an interfering RNA, the administration may in particular be carried out once every 2 to 8 weeks, preferably with 1 to 10 mg/Kg of interfering RNA, in combination with a pharmaceutically acceptable excipient.

The invention also concerns an in vitro method for determining, on the basis of a biological sample, the early diagnosis of gastric cancer, characterized in that the expression of the gene encoding IL-17A, IL-17F, IL-17RA and/or IL-17RC is determined.

The measurement of the expression of the gene encoding IL-17A, IL-17F, IL-17RA and/or IL-17RC comprises the following steps:
a. biological material is extracted from the biological sample;
b. the biological material is brought into contact with at least one reagent specific for the gene encoding IL-17A, IL-17F, IL-17RA and/or IL-17RC;
c. the expression of the gene encoding IL-17A, IL-17F, IL-17RA and/or IL-17RC is determined.

The biological material extracted during step a) may comprise nucleic acids or proteins.

Said specific reagent of step b) may comprise a hybridization probe or an antibody specific for the gene encoding IL-17A, IL-17F, IL-17RA and/or IL-17RC.

The invention also concerns the use of at least one reagent specific for the gene encoding IL-17A, IL-17F, IL-17RA and/or IL-17RC for determining the early diagnosis of gastric cancer.

The invention also concerns a kit for early diagnosis of gastric cancer, comprising at least one reagent specific for the gene encoding IL-17A, IL-17F, IL-17RA and/or IL-17RC.

The analysis of the expression of the IL-17A, IL-17F, IL-17RA and/or IL-17RC genes then makes it possible to have a tool for diagnosing gastric cancer. It is, for example, possible to analyze the expression of the target gene in a patient liable to develop gastric cancer, and to compare with known average expression values for the target gene of patients suffering from gastric cancer and known average expression values for the target gene of healthy patients.

For the purpose of the present invention, the term “biological sample” is intended to mean any sample taken from a patient, and which may contain a biological material as defined hereinafter. This biological sample may in particular be a blood, serum or tissue sample from the patient. This biological sample is provided by any means of sampling known to those skilled in the art. According to one preferred embodiment of the invention, the biological sample taken from the patient is a blood sample.

During step a) of the method according to the invention, the biological material is extracted from the biological sample by any of the protocols for extracting and purifying nucleic acids or proteins known to those skilled in the art.

For the purpose of the present invention, the term “biological material” is intended to mean any material which makes it possible to detect the expression of a target gene. The biological material may comprise in particular proteins, or nucleic acids such as, in particular, deoxyribonucleic acids (DNA) or ribonucleic acids (RNA). The nucleic acid may in particular be an RNA (ribonucleic acid). According to one preferred embodiment of the invention, the biological material extracted during step a) comprises nucleic acids, preferably RNAs, and even more preferably total RNA. Total RNA comprises transfer RNAs, messenger RNAs (mRNAs), such as the mRNAs transcribed from the target gene, but also transcribed from any other gene, and ribosomal RNAs. This biological material comprises material specific for a target gene, such as, in particular, the mRNAs transcribed from the target gene or the proteins derived from these mRNAs, but may also comprise material not specific for a target gene, such as, in particular, the mRNAs transcribed from a gene other than the target gene, the tRNAs, or the rRNAs derived from genes other than the target gene.

By way of indication, the nucleic acid extraction can be carried out by means of:
a step of lysis of the cells present in the biological sample, in order to release the nucleic acids contained in the patient's cells. By way of example, use may be made of the lysis methods as described in patent applications WO 00/05338, WO 99/53304 and WO 99/15321. Those skilled in the art may use other well-known methods of lysis, such as heat shock or osmotic shock or chemical lyses with chaotropic agents such as guanidium salts (U.S. Pat. No. 5,234,809);
a step of purification, for separating the nucleic acids from the other cell constituents released in the lysis step. This step generally makes it possible to concentrate the nucleic acids, and can be adapted to the purification of DNA or of RNA. By way of example, it is possible to use magnetic particles optionally coated with oligonucleotides, by adsorption or covalence (in this respect, see U.S. Pat. No. 4,672,040 and U.S. Pat. No. 5,750,338), and thus to purify the nucleic acids which are bound to these magnetic particles, by means of a washing step. This nucleic acid purification step is particularly advantageous if it is desired to subsequently amplify said nucleic acids. One particularly advantageous embodiment of these magnetic particles is described in patent applications: WO-A-97/45202 and WO-A-99/35500. Another advantageous example of a nucleic acid purification method is the use of silica, either in the form of a column, or in the form of inert or magnetic particles. Other, very widely used, methods are based on ion exchange resins in a column or in a paramagnetic particulate format. Another very relevant but nonexclusive method for the invention is that of adsorption onto a metal oxide support.

In the case of the extraction of proteins, the first step generally comprises, as for the nucleic acids, lysis of the cells. An osmotic shock may be sufficient to rupture the cell membrane of fragile cells, it being possible for said osmotic shock to be carried out in the presence of a detergent. A mechanic action may also be added to the process (piston homogenizer, for example). The lysis may also be induced by ultrasound, or by mechanical lysis using glass beads. The extraction of the proteins of interest can subsequently be carried out by chromatography, such as, in particular, on a gel chromatography column, packed with a resin comprising hollow, porous beads. The pore size of these beads is such that the proteins are separated according to their size. Mention may also be made of ion exchange column chromatography, which enables proteins to be extracted according to their electrostatic affinity with respect to charged groups of the resin.

During step b), and for the purpose of the present invention, the term “specific reagent” is intended to mean a reagent which, when it is brought into contact with biological material as defined above, binds with the material specific for said target gene.

By way of indication, when the specific reagent and the biological material are of nucleic origin, bringing the specific reagent into contact with the biological material enables the specific reagent to hybridize with the material specific for the target gene. The term “hybridization” is intended to mean the process during which, under suitable conditions, two nucleotide fragments bind to one another with stable, specific hydrogen bonds so as to form a double-stranded complex. These hydrogen bonds form between the complementary bases adenine (A) and thymine (T) (or uracil (U)) (this is then referred to as an A-T bond) or between the complementary bases guanine (G) and cytosine (C) (this is then referred to as a G-C bond). The hybridization of two nucleotide fragments may be total (reference is then made to complementary nucleotide fragments or sequences), i.e. the double-stranded complex obtained during this hybridization comprises only A-T bonds and C-G bonds. This hybridization may be partial (reference is then made to sufficiently complementary nucleotide fragments or sequences), i.e. the double-stranded complex obtained comprises A-T bonds and C-G bonds allowing the double-stranded complex to form, but also bases not bonded to a complementary base. The hybridization between two nucleotide fragments depends on the working conditions which are used, and in particular on the stringency. The stringency is defined in particular according to the base composition of the two nucleotide fragments, and also by the degree of mismatching between two nucleotide fragments. The stringency may also depend on the reaction parameters, such as the concentration and the type of ionic species present in the hybridization solution, the nature and the concentration of denaturing agents and/or the hybridization temperature. All these data are well known and the appropriate conditions can be determined by those skilled in the art. In general, according to the length of the nucleotide fragments that it is desired to hybridize, the hybridization temperature is between approximately 20 and 70° C., in particular between 35 and 65° C., in a saline solution at a concentration of approximately 0.5 to 1 M. A sequence, or nucleotide fragment, or oligonucleotide, or polynucleotide, is a series of nucleotide motifs assembled together by phosphoric ester bonds, characterized by the informational sequence of the natural nucleic acids, capable of hybridizing to a nucleotide fragment, it being possible for the series to contain monomers of different structures and to be obtained from a natural nucleic acid molecule and/or by genetic recombination and/or by chemical synthesis. A motif is derived from a monomer which may be a natural nucleotide of a nucleic acid, the constitutive elements of which are a sugar, a phosphate group and a nitrogenous base; in DNA, the sugar is deoxy-2-ribose, in RNA, the sugar is ribose; depending on whether it is a question of DNA or RNA, the nitrogenous base is chosen from adenine, guanine, uracil, cytosine and thymine; or alternatively the monomer is a nucleotide modified in at least one of the three constitutive elements; by way of example, the modification may occur either at the level of the bases, with modified bases such as inosine, methyl-5-deoxycytidine, deoxyuridine, dimethylamino-5-deoxyuridine, diamino-2,6-purine, bromo-5-deoxyuridine or any other modified base capable of hybridization, or at the level of the sugar, for example the replacement of at least one deoxyribose with a polyamide, or else at the level of the phosphate group, for example replacement thereof with esters chosen in particular from diphosphates, alkyl and aryl phosphonates and phosphorothioates.

According to one particular embodiment of the invention, the specific reagent comprises at least one amplification primer. For the purpose of the present invention, the term “amplification primer” is intended to mean a nucleotide fragment comprising from 5 to 100 nucleic motifs, preferably from 15 to 30 nucleic motifs, allowing the initiation of an enzymatic polymerization, such as, in particular, an enzymatic amplification reaction. The term “enzymatic amplification reaction” is intended to mean a process generating multiple copies of a nucleotide fragment through the action of at least one enzyme. Such amplification reactions are well known to those skilled in the art and mention may in particular be made of the following techniques:
PCR (Polymerase Chain Reaction), as described in U.S. Pat. No. 4,683,195, U.S. Pat. No. 4,683,202 and U.S. Pat. No. 4,800,159,
LCR (Ligase Chain Reaction), disclosed, for example, in patent application EP 0 201 184,
RCR (Repair Chain Reaction), described in patent application WO 90/01069,
3SR (Self Sustained Sequence Replication) with patent application WO 90/06995,
NASBA (Nucleic Acid Sequence-Based Amplification) with patent application WO 91/02818, and
TMA (Transcription Mediated Amplification) with U.S. Pat. No. 5,399,491.

When the enzymatic amplification is a PCR, the specific reagent comprises at least 2 amplification primers, specific for the target gene, in order to allow the amplification of the target-gene-specific material. The target-gene-specific material then preferably comprises a complementary DNA obtained by reverse transcription of messenger RNA derived from the target gene (reference is then made to target-gene-specific cDNA) or a complementary RNA obtained by transcription of the cDNAs specific for a target gene (reference is then made to target-gene-specific cRNA). When the enzymatic amplification is a PCR carried out after a reverse transcription reaction, this is referred to as RT-PCR.

According to another preferred embodiment of the invention, the specific reagent of step b) comprises at least one hybridization probe.

The term “hybridization probe” is intended to mean a nucleotide fragment comprising at least 5 nucleotide motifs, for instance from 5 to 100 nucleic motifs, in particular from 10 to 35 nucleic motifs, having a hybridization specificity under given conditions so as to form a hybridization complex with the material specific for a target gene. In the present invention, the target-gene-specific material may be a nucleotide sequence included in a messenger RNA derived from the target gene (reference is then made to target-gene-specific mRNA), a nucleotide sequence included in a complementary DNA obtained by reverse transcription of said messenger RNA (reference is then made to target-gene-specific cDNA), or else a nucleotide sequence included in a complementary RNA obtained by transcription of said cDNA as described above (reference will then be made to target-gene-specific cRNA). The hybridization probe may comprise a label for its detection. The term “detection” is intended to mean either a direct detection by a physical method, or an indirect detection by a detection method using a label. Many detection methods exist for detecting nucleic acids [see, for example, Kricka et al., Clinical Chemistry, 1999, n^{o} 45(4), p. 453-458 or Keller G. H. et al., DNA Probes, 2nd Ed., Stockton Press, 1993, sections 5 and 6, p. 173-249]. The term “label” is intended to mean a tracer capable of generating a signal that can be detected. A nonlimiting list of these tracers includes enzymes which produce a signal detectable, for example, by colorimetry, fluorescence or luminescence, such as horseradish peroxydase, alkaline phosphatase, beta-galactosidase, glucose-6-phosphate dehydrogenase; chromophores such as fluorescents, luminescent or dye compounds; electron-dense groups detectable by electron microscopy or by their electrical properties such as conductivity, by amperometry or voltametry methods, or by impedance measurements; groups that can be detected by optical methods such as diffraction, surface plasmon resonance, contact angle variation or by physical methods such as atomic force spectroscopy, tunnel effect, etc.; radioactive molecules such as ³²P, ³⁵S or ¹²⁵I.

For the purpose of the present invention, the hybridization probe may be a “detection” probe. In this case, the “detection” probe is labeled with a label as defined above. The detection probe may in particular be a “molecular beacon” detection probe as described by Tyagi & Kramer (Nature biotech, 1996, 14:303-308). These “molecular beacons” become fluorescent during hybridization. They have a stem-loop structure and contain a fluorophore and a quencher group. The binding of the specific loop sequence with its complementary target nucleic acid sequence causes the stem to uncoil and a fluorescent signal to be emitted during excitation at the appropriate wavelength.

For the detection of the hybridization reaction, use may be made of target sequences that have been labeled directly (in particular by the incorporation of a label within the target sequence) or indirectly (in particular using a detection probe as defined above) the target sequence. A step for labeling and/or cleaving the target sequence can in particular be carried out before the hybridization step, for example using a labeled deoxyribonucleotide triphosphate during the enzymatic amplification reaction. The cleavage can be carried out in particular through the action of imidazole and manganese chloride. The target sequence can also be labeled after the amplification step, for example by hybridizing a detection probe according to the sandwich hybridization technique described in document WO 91/19812. Another particular preferred method for labeling nucleic acids is described in application FR 2 780 059. According to one preferred embodiment of the invention, the detection probe comprises a fluorophore and a quencher.

The hybridization probe may also be a “capture” probe. In this case, the “capture” probe is immobilized or immobilizable on a solid support by any appropriate means, i.e. directly or indirectly, for example by covalence or adsorption. As solid support, use may be made of synthetic materials or natural materials, optionally chemically modified, in particular polysaccharides such as cellulose-based materials, for example paper, cellulose derivatives such as cellulose acetate and nitrocellulose, or dextran, polymers, copolymers, in particular based on styrene-type monomers, natural fibers such as cotton, and synthetic fibers such as nylon; mineral materials such as silica, quartz, glasses, ceramics; latices; magnetic particles; metal derivatives, gels, etc. The solid support may be in the form of a microtitration plate, of a membrane as described in application WO-A-94/12670, or of a particle. These steps of hybridization on a support may be preceded by an enzymatic amplification reaction step, as defined above, in order to increase the amount of target genetic material.

By way of indication, when the specific reagent and the biological material are of protein origin, bringing the specific reagent and the biological material into contact allows the formation of an “antigen-antibody” complex between the specific reagent and target-gene-specific material.

During step c), the determination of the expression of the target gene can be carried out by any of the protocols known to those skilled in the art.

In general, the expression of a target gene can be analyzed by detection of the mRNAs (messenger RNAs) which are transcribed from the target gene at a given instant or by the detection of the proteins derived from these mRNAs.

The invention preferentially concerns the determination of the expression of a target gene by detection of the mRNAs derived from this target gene.

When the specific reagent comprises one or more amplification primers, it is possible, during step c) of the method according to the invention, to determine the expression of a target gene in the following way:

1) after having extracted, as biological material, the total RNA (comprising the transfer RNAs (tRNAs), the ribosomal RNAs (rRNAs) and the messenger RNAs (mRNAs)) of a biological sample as presented above, a reverse transcription step is carried out in order to obtain the complementary DNAs (or cDNAs) of said mRNAs. By way of indication, this reverse transcription reaction can be carried out using a reverse transcriptase enzyme which makes it possible to obtain, from an RNA fragment, a complementary DNA fragment. The reverse transcriptase enzyme originating from AMV (Avian Myoblastosis Virus) or from MMLV (Moloney Murine Leukemia Virus) can in particular be used. When it is more particularly desired to obtain only the cDNAs of the mRNAs, this reverse transcription step is carried out in the presence of nucleotide fragments comprising only thymine bases (polyT), which hybridize by complementarity on the polyA sequence of the mRNAs so as to form a polyT-polyA complex which then serves as a starting point for the reverse transcription reaction carried out by the reverse transcriptase enzyme. cDNAs complementary to the mRNAs derived from a target gene (target-gene-specific cDNA) and cDNAs complementary to the mRNAs derived from genes other than the target gene (cDNAs not specific for the target gene) are then obtained;
2) the amplification primer(s) specific for a target gene is (are) brought into contact with the target-gene-specific cDNAs and the cDNAs not specific for the target gene. The amplification primer(s) specific for a target gene hybridize(s) with the target-gene-specific cDNAs and a predetermined region, of known length, of the cDNAs originating from the mRNAs derived from the target gene is specifically amplified. The cDNAs not specific for the target gene are not amplified, whereas a large amount of target-gene-specific cDNAs is then obtained. For the purpose of the present invention, reference is made, without distinction, to “target-gene-specific cDNAs” or to “cDNAs originating from the mRNAs derived from the target gene”. This step can be carried out in particular by a PCR-type amplification reaction or by any other amplification technique as defined above;
3) the expression of the target gene is determined by detecting and quantifying the target-gene-specific cDNAs obtained during step 2) above. This detection can be carried out after electrophoretic migration of the target-gene-specific cDNAs according to their size. The gel and the migration medium can include ethidium bromide so as to allow direct detection of the target-gene-specific cDNAs when the gel is placed, after a given migration period, on a UV (ultraviolet)-ray light table, through the emission of a light signal. The greater the amount of target-gene-specific cDNAs, the brighter this light signal. These electrophoresis techniques are well known to those skilled in the art. The target-gene-specific cDNAs can also be detected and quantified using a quantification range obtained by means of an amplification reaction carried out until saturation. In order to take into account the variability of enzymatic efficiency that may be observed during the various steps (reverse transcription, PCR, etc.), the expression of a target gene of various groups of patients can be standardized by simultaneously determining the expression of a “housekeeping” gene, the expression of which is similar in the various groups of patients. By realizing a ratio of the expression of the target gene to the expression of the housekeeping gene, i.e. by realizing a ratio of the amount of target-gene-specific cDNAs to the amount of housekeeping-gene-specific cDNAs, any variability between the various experiments is thus corrected. Those skilled in the art may refer in particular to the following publications: Bustin S A, J Mol Endocrinol, 2002, 29: 23-39; Giulietti A Methods, 2001, 25: 386-401.

When the specific reagent comprises at least one hybridization probe, the expression of a target gene can be determined in the following way:

1) after having extracted, as biological material, the total RNA of a biological sample as presented above, a reverse transcription step is carried out as described above in order to obtain cDNAs complementary to the mRNAs derived from a target gene (target-gene-specific cDNA) and cDNAs complementary to the mRNAs derived from genes other than the target gene (cDNA not specific for the target gene);
2) all the cDNAs are brought into contact with a support, on which are immobilized capture probes specific for the target gene whose expression it is desired to analyze, in order to carry out a hybridization reaction between the target-gene-specific cDNAs and the capture probes; the cDNAs not specific for the target gene do not hybridize to the capture probes. The hybridization reaction can be carried out on a solid support which includes all the materials as indicated above. According to one preferred embodiment, the hybridization probe is immobilized on a support. The hybridization reaction may be preceded by a step of enzymatic amplification of the target-gene-specific cDNAs, as described above, so as to obtain a large amount of target-gene-specific cDNAs and to increase the probability of a cDNA specific for a target gene hybridizing to a capture probe specific for the target gene. The hybridization reaction may also be preceded by a step for labeling and/or cleaving the target-gene-specific cDNAs, as described above, for example using a labeled deoxyribonucleotide triphosphate for the amplification reaction. The cleavage can be carried out in particular through the action of imidazole and manganese chloride. The target-gene-specific cDNA can also be labeled after the amplification step, for example by hybridizing a labeled probe according to the sandwich hybridization technique described in document WO-A-91/19812. Other particular preferred methods for labeling and/or cleaving nucleic acids are described in applications WO 99/65926, WO 01/44507, WO 01/44506, WO 02/090584 and WO 02/090319;
3) a step for detection of the hybridization reaction is subsequently carried out. The detection can be carried out by bringing the support, on which the target-gene-specific capture probes are hybridized with the target-gene-specific cDNAs, into contact with a “detection” probe labeled with a label, and detecting the signal emitted by the label. When the target-gene-specific cDNA has been labeled beforehand with a label, the signal emitted by the label is detected directly.

When the at least one specific reagent brought into contact in step b) of the method according to the invention comprises at least one hybridization probe, the expression of a target gene can also be determined in the following way:

1) after having extracted, as biological material, the total RNA of a biological sample as presented above, a reverse transcription step is carried out as described above in order to obtain the cDNAs of the mRNAs of the biological material. The polymerization of the complementary RNA of the cDNA is subsequently carried out using a T7 polymerase enzyme which functions under the control of a promoter and which makes it possible to obtain, from a DNA template, the complementary RNA. The cRNAs of the cDNAs of the mRNAs specific for the target gene (reference is then made to target-gene-specific cRNA) and the cRNAs of the cDNAs of the mRNAs not specific for the target gene are then obtained;
2) all the cRNAs are brought into contact with a support on which are immobilized capture probes specific for the target gene whose expression it is desired to analyze, in order to carry out a hybridization reaction between the target-gene-specific cRNAs and the capture probes; the cRNAs not specific for the target gene do not hybridize to the capture probes. The hybridization reaction can also be preceded by a step for labeling and/or cleaving the target-gene-specific cRNAs, as described above;
3) a step for detecting the hybridization reaction is subsequently carried out. The detection can be carried out by bringing the support, on which the target-gene-specific capture probes are hybridized with the target-gene-specific cRNA, into contact with a “detection” probe labeled with a label, and detecting the signal emitted by the label. When the target-gene-specific cRNA has been labeled beforehand with a label, the signal emitted by the label is detected directly. The use of cRNA is particularly advantageous when a support of the biochip type on which a large number of probes are hybridized is used.

According to one particular embodiment of the invention, steps B and C are carried out at the same time. This preferred method can in particular be carried out by “real time NASBA”, which groups together, in a single step, the NASBA amplification technique and real time detection which uses “molecular beacons”. The NASBA reaction takes place in the tube, producing the single-stranded RNA with which the specific “molecular beacons” can simultaneously hybridize to give a fluorescent signal. The formation of the new RNA molecules is measured in real time by continuous verification of the signal in a fluorescent reader.

By way of indication, when the specific reagent and the biological material are of protein origin, step c) can in particular be carried out by Western blotting or ELISA, or any other method known to those skilled in the art.

By way of indication, the ELISA technique is a reference biochemical technique used in immunology for detecting the presence of an antibody or of an antigen in a sample. The technique uses two antibodies, one of them being specific to the antigen and the other being coupled to an enzyme.

By way of indication, the Western blotting technique is a test for detecting a specific protein in a sample using an antibody specific for this protein, comprising the following steps:

The first step is a gel electrophoresis, which makes it possible to separate the proteins from the sample according to their size.

The proteins in the gel are then transferred onto a membrane (nitrocellulose, PVDF, etc.) by pressure or by application of an electric current, the proteins attaching to the membrane by virtue of hydrophobic and ionic interactions.

After saturation of the nonspecific interaction sites, a first antibody, specific for the protein to be studied (primary antibody), is incubated with the membrane.

The membrane is subsequently rinsed in order to remove the unbound primary antibodies, and then incubated with “secondary” antibodies, which will bind to the primary antibodies. This secondary antibody is normally bonded to an enzyme which allows visual identification of the protein studied on the membrane.

As for the ELISA techniques, the addition of a substrate for the enzyme generates a colored reaction which is visible on the membrane.

The figures will make it possible to understand the invention more clearly.

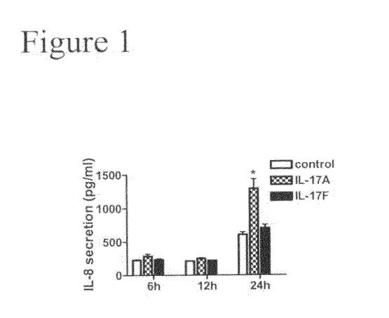

FIG. 1 shows the effects of IL-17A and IL-17F on IL-8 secretion by AGS cells. The AGS cells were cultured in the presence or absence of 50 ng/ml of rhIL-17A or rhIL-17F for 6, 12 or 24 h. The amount of IL-8 in the supernatant was measured by ELISA. The results are given as mean±SEM on 4 series of experiments. *P<0.05, compared to the control group.

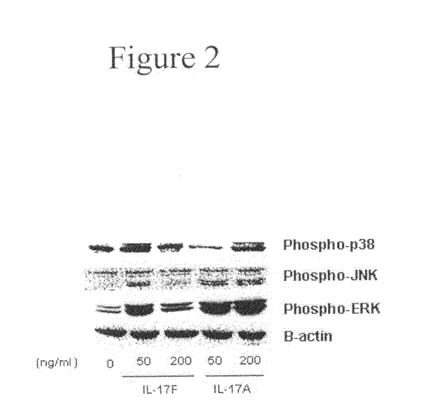

FIG. 2 shows the effects of IL-17A and IL-17F on the activation of MAPKs (mitogen-activated protein kinases) in AGS cells. A Western blotting analysis of phosphorylated p38, ERK and JNK was carried out using AGS cells stimulated with IL-17A or IL-17F at a concentration of 50 or 200 ng/ml for 30 minutes. The results are representative of 3 independent series of experiments.

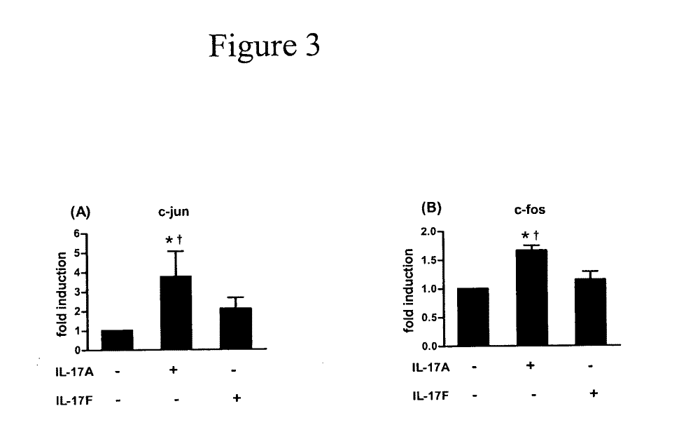

FIG. 3 shows the effects of IL-117A and IL-17F on the activation of AP-1 in AGS cells. The AGS cells were cultured in serum-depleted medium for 48 h and then stimulated, at a concentration of 50 ng/ml, with IL-17A or with IL-17F for 20 min. The DNA-binding activity of the transcription factors c-jun and c-fos was analyzed using the TransAM™ AP-1 kit. The results are given as mean±SEM obtained on triplicates. *P<0.05, compared to the untreated control cells. ^{†}P<0.05, compared to the IL-117F-stimulated cells.

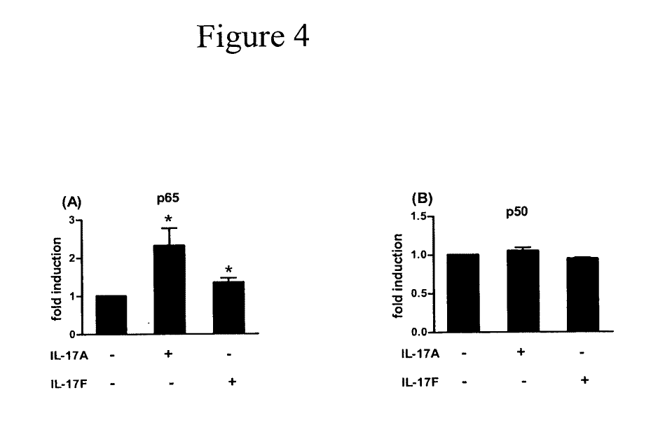

FIG. 4 shows the effects of IL-17A and IL-17F on the activation of NFκB in AGS cells. The AGS cells were cultured in serum-depleted medium for 48 h and then stimulated, at a concentration of 50 ng/ml, with IL-17A or IL-17F for 20 min. The DNA-binding activity of the transcription factors p65 and p55 was analyzed using the TransAM™ NFκB kit. The results are given as mean±SEM obtained on triplicates. *P<0.05, compared to the untreated control cells.

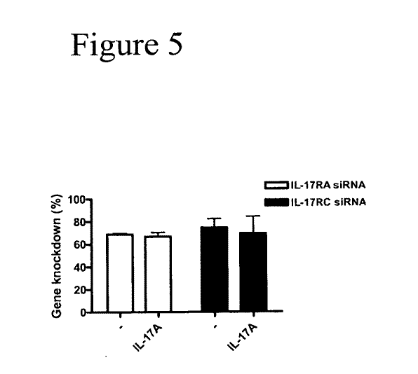

FIG. 5 shows the inhibition of the gene encoding IL-17R (A) and IL17-RC (B) by interfering RNA (iRNA). The AGS cells (1×10⁵) were transfected with 0.5 μg of IL-17R iRNA, IL-17RC iRNA or control iRNA. The expression of IL-17R mRNA and IL-17RC mRNA, standardized with GAPDH, was analyzed 36 h after transfection, by quantitative RT-PCR. The results are given as mean±SEM obtained on duplicates. *P<0.05, compared with the cells transfected with control iRNA.

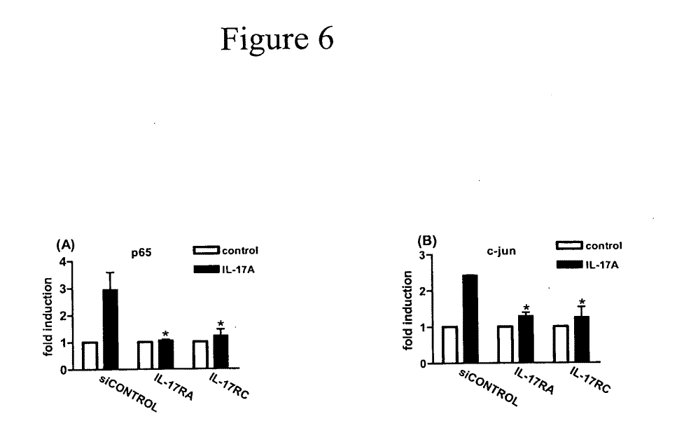

FIG. 6 shows the effects of the inhibition of the genes encoding IL-17R and IL-17RC on the DNA-binding activity of p65 NFκB (A) and c-jun AP-1 (B). The AGS cells (1×10⁵) were transfected with 0.5 μg of IL-17R iRNA, IL-17RC iRNA or control siRNA. 24 h after transfection, the cells were placed in serum-depleted medium for 24 h, and then treated with 50 ng/ml of IL-17A or of IL-17F. The DNA-binding activity of p-65 (A) was analyzed using a TransAM™ NFκB kit. The expression of c-Jun mRNA (B) was analyzed by quantitative RT-PCR 30 min after the stimulation. The results are given as mean±SEM obtained on duplicates. *P<0.05, compared with the cells transfected with control iRNA.

The following examples are given by way of illustration and are no way limiting in nature. They will make it possible to understand the invention more clearly.

Cell lines. A human gastric cancer cell line (AGS) of the ATCC collection was used and cultured (37° C., 5% CO₂) in DMEM medium (Dulbecco's Modified Eagle's Medium, Invitrogen Life Technologies, Carlsbad, Calif.) supplemented with fetal calf serum (10% v/v).

IL-8 ELISA. The AGS cells were stimulated with recombinant human IL-17A or IL-17F (rhIL-17A, rhIL-17F, R&D Systems, USA) at a concentration of 50 ng/ml for 12 h or 24 h. The amount of IL-8 secreted by the cells in culture was determined by ELISA (Diaclone, France).

Western blotting. The AGS cells were cultured in serum-depleted DMEM medium for 24 h, and then treated with IL-17A or IL-17F at a concentration of 50 ng/ml or 200 ng/ml for 30 minutes. The MAPK activation was analyzed by Western blotting in the presence or absence of IL-17A and IL-17F. For this, the cells were lysed in a lysis medium (20 mM Hepes, pH 7.7, 2.5 mM MgCl₂, 0.1 mM EDTA, 20 mM β-glycerophosphate, 100 mM NaCl, 0.05% Triton X100, 0.5 mM DTT, 0.1 mM Na₃VO₄, 20 μg/ml leupeptin, 20 μg/ml aprotinin, 100 μg/ml PMSF), and the protein concentration was determined using a BCA kit (PIERCE, USA). Aliquots containing 80 μg of proteins were used in electrophoresis on a gel comprising 10% of SDS-polyacrylamide. After electrophoretic separation, the proteins were transferred from the gel to a nitrocellulose membrane (Amersham, USA). The membranes were subsequently saturated (5% powdered milk, 0.01% Tween20, 2% fetal calf serum) for 1 h, then washed 3 times in PBS medium containing 0.1% Tween20. The membranes were subsequently brought into contact with antibodies against phospho-p38, phospho-ERK, phospho-JNK (Cell Signaling Technology, USA) or β-actin (Chemicon, USA).

Transcription factor activation. The AGS cells were cultured in a serum-depleted DMEM medium for 24 h, and then treated with IL-17A or IL-17F at a concentration of 50 ng/ml for 1 h. The nuclear protein extracts were obtained from the cell lines using a kit (Active Motif, USA). The DNA-binding activity of the C-jun, c-fos or p65, p55 factors was analyzed using a TransAM™ AP-1 or TransAM™ NFkB kit (Active Motif, CA, USA).

Quantitative analysis by RT-PCR. The AGS cells were cultured in a serum-depleted DMEM medium for 24 h, and then treated with IL-17A or IL-17F at a concentration of 50 ng/ml for 30 minutes. The RNAs were extracted from the AGS cells with TRIzol (Invitrogen, USA), and 1 μg of RNA was reverse transcribed using the Superscript reverse transcription system (Invitrogen, USA). The PCR amplification was carried out on a LightCycler (Roche) using the Fast-Start™ DNA Master SYBR Green I real-time PCR kit (Roche Molecular Biochemicals). GAPDH was used as housekeeping gene. The C-Jun and GAPDH primers were purchased from Search-LC GmbH (Heidelberg, Germany). The thermocycle was carried out using a volume of 20 μl containing the primers at a concentration of 10 μM, 25 mM of magnesium chloride (MgCl₂), Taq and SYBR Green I dye as recommended in the LightCycler Fast start DNA Master SYBR green I kit (Roche). The primers for the IL-17RA (Genbank accession No. NM_{—}014339) and IL-17RC (Genbank accession No. NM_{—}153460) were obtained from Eurogentec (San Diego, Calif.): (IL-17RA forward: SEQ ID No. 1 5′-AGACACTCCAGAACCAATTCC-3′, IL-17RA reverse: SEQ ID No. 2 5′-TCTTAG AGTTGCTCTCCACCA-3′; IL-17RC forward: SEQ ID No. 3 5′-ACCAGAACCTCTGGCAAGC-3′, IL-17RC reverse: SEQ ID No. 4 5′-GAGCTGTTCACCTGAACACA-3′). The PCR comprised 45 amplification cycles (10 seconds at 95° C., 10 seconds at 68° C. and 16 seconds at 72° C.). The results were expressed by the target gene/GAPDH mRNA ratio.

Interfering RNAs. Interfering RNAs corresponding to nucleotides 1623-1641 of human IL-17RA (NM_{—}014339) and to nucleotides 985-1003 of human IL-17RC (NM_{—}153460) were obtained from Dharmacon (Lafayette, Colo., USA). A control iRNA was also used (siCONTROL). The AGS cells (1×10⁵) were transfected with 0.5 μg of IL-17RA iRNA, of IL-17RC iRNA or of control iRNA using a Nucleofector kit (Amaxa GmbH, Cologne, Germany). 24 h after transfection, the cells were cultured in a serum-depleted medium for 12 h, and then treated with 50 ng/ml of IL-17A. The expression of C-Jun mRNA, 30 min after stimulation, was determined by quantitative RT-PCR as described above. The p65 DNA-binding activity, 1 h after stimulation, was analyzed using the TransAM™ NFkB kit.

Statistical analysis. The results were expressed as mean±SEM. The statistical values of the differences were determined using the Student's t-test and the differences resulting in a value P<0.05 were considered to be statistically significant.

### Results

### Effects of IL-17A and IL-17F on IL-8 Production by AGS Cells

The results are shown in FIG. 1. Stimulation of the AGS cells with IL-17A for 24 h increased the basal expression of IL-8, which is an interleukin involved in neutrophil recruitment, from 604.3±39.2 pg/ml to 1290.8±142.6 pg/ml, (P<0.05).

### Effects of IL-17A and IL-17F on MAPK Activation in AGS Cells

The results are shown in FIG. 2. IL-17A or IL-17F induced phosphorylation of the various three MAPKs, involved in the development of cancer. The phosphorylation of ERK or JNK via IL-17A or IL-17F was comparable. IL-17F induced more p38 phosphorylation than IL-17A.

Effects of IL-17A and IL-17F on c-fos and c-jun Activation in AGS Cells

The results are shown in FIG. 3. IL-17A and IL-17F induced an increase in the DNA-binding activity of c-jun and c-fos, involved in the development of cancer. IL-17A induced a 3.8-fold (p<0.05) and 1.7-fold (p<0.05) increase for c-jun and c-fos, respectively. IL-17F induced a 2-fold (p=0.07) and 1.2-fold (p=0.37) increase for c-jun and c-fos.

### Effects of IL-17A and IL-17F on NFkB Activation in AGS Cells

The results are shown in FIG. 4. In the AGS cells stimulated with IL-17A, the DNA-binding activity of p65, involved in the development of cancer, was 1.6 times (p<0.05) higher than in the nonstimulated cells. The stimulation with IL-17F induced an induction of p65 (p<0.05).

### Effects of the Inhibition of the Genes Encoding IL-17RA and IL-17RC on the DNA-Binding Activity of NFκB and AP-1 in AGS Cells

The expression of IL-17RA and IL-17RC was greatly reduced by the transfection of IL-17RA iRNA and IL-17RC iRNA (FIG. 5). The underexpression of IL-17RA induced a reduction in the activation of p65 and c-Jun, induced by IL-17A, inducing a 97% inhibition (p<0.05) and 80% inhibition (p<0.01), respectively, by comparison with the cells transfected with a control iRNA. Similarly, the underexpression of IL-17RC induced an 89% inhibition (p<0.05) and an 82% inhibition (p<0.05) of the activation of p65 and c-Jun, respectively (FIG. 6).

All these results demonstrate that IL-17A and IL-17F, and the IL-17RA and IL-17RC receptors, are therapeutic targets for combating gastric cancer.

1. A method for the manufacture of a medicament for inhibiting, preventing or treating gastric cancer, comprising providing at least one interleukin-17 inhibitor and/or of at least one IL-17 receptor inhibitor.

2. The method claimed in claim 1, according to which the interleukin-17 is interleukin-17A (IL-17A) or interleukin-17F (IL-17F).

3. The method claimed in claim 1, according to which said IL-17 receptor is the IL-17 receptor A or the IL-17 receptor C.

4. The method claimed in claim 1, according to which the interleukin-17 inhibitor is an antibody directed against IL-17, and/or the inhibitor of said IL-17 receptor is an antibody directed against the IL-17 receptor.

5. The method claimed in claim 1, according to which the interleukin-17 inhibitor is an interfering RNA against IL-17, and/or the inhibitor of said IL-17 receptor is an interfering RNA against the IL-17 receptor.

6. A pharmaceutical composition comprising, as active substance, at least one interleukin-17 inhibitor and/or at least one IL-17 receptor inhibitor in combination with a pharmaceutically appropriate carrier.

7. The composition as claimed in claim 6, wherein the interleukin-17 is interleukin-17A (IL-17A) or interleukin 17F (IL-17F).

8. The composition as claimed in claim 6, wherein said IL-17 receptor is the IL-17 receptor A or the IL-17 receptor C.

9. The composition as claimed in claim 6, wherein the interleukin-17 inhibitor is an antibody directed against IL-17, and/or the inhibitor of said IL-17 receptor is an antibody directed against the IL-17 receptor.

10. The composition as claimed in claim 8, wherein the interleukin-17 inhibitor is an interfering RNA against IL-17, and/or the inhibitor of said IL-17 receptor is an interfering RNA against the IL-17 receptor.

11. A method for inhibiting, preventing or treating gastric cancer, comprising providing the pharmaceutical composition as claimed in claim 8.


SEQUENCE LISTING (4 sequences)

SEQ ID NO: 1 - DNA, 21 bp, Homo sapiens
agacactccagaaccaattcc

SEQ ID NO: 2 - DNA, 21 bp, Homo sapiens
tcttagagttgctctccacca

SEQ ID NO: 3 - DNA, 19 bp, Homo sapiens
accagaacctctggcaagc

SEQ ID NO: 4 - DNA, 20 bp, Homo sapiens
gagctgttcacctgaacaca